(19) United States
(12) Patent Application Publication
(10) Pub. No.: US 2007/0237823 A1
(43) Pub. Date: Oct. 11, 2007

(54) Solid Pharmaceutical Form Comprising and Ltb4 Antagonist

(76) Inventors: Thomas Bock, Dully (CH); Thomas Friedl, Ochsenhausen (DE); Thomas Hantke, Heidelberg (DE); Joerg Neumann, Kirchhain (DE); Joerg Rosenberg, Elberstadt (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/630,212

(22) PCT Filed: Apr. 26, 2005
(86) PCT No.: PCT/EP05/04443

§ 371 Date: Dec. 20, 2006

(30) Foreign Application Priority Data
May 4, 2004 (EP) 04010535,5

(51) Int. Cl. A61K 9/10; A61P 11/06; A61P 17/06; A61P 19/02; A61P 25/08; A61P 9/10

(52) U.S. Cl. 424/486

(57) ABSTRACT

The invention relates to a solid pharmaceutical form obtainable by melt extrusion comprising an LTB4 antagonist, which is embedded in a polymer matrix (solid dispersion).

### BACKGROUND OF THE INVENTION

The invention relates to a solid pharmaceutical form obtainable by melt extrusion comprising an LTB₄ antagonist, which is embedded in a polymer matrix (solid dispersion).

LTB₄ antagonists which contain a benzamidine group are compounds with pharmacologically valuable properties. LTB₄ antagonists may provide great therapeutic benefit, for example, in the treatment of treat arthritis, asthma, chronic obstructive lung diseases, psoriasis, ulcerative colitis, Alzheimer's disease, shock, reperfusion damage/ischaemia, cystic fibrosis, atherosclerosis and multiple sclerosis.

Such compounds are known e.g. from International Patent Applications WO 93/16036, WO 94/11341, WO 96/02497, WO 97/21670, WO 98/11062, WO 98/11119, WO 01/25186 and WO 01/51457.

The U.S. patent application U.S. Pat. No. 4,801,460 describes a process for producing solid pharmaceutical forms by extruding polymer melts which contain active ingredients, the polymers used being homo- or copolymers of N-vinylpyrrolidone. However, there is no hint to LTB₄ antagonists.

The International patent application WO 03/007922 discloses a tablet comprising an LTB₄ antagonist and a wetting agent, in particular lauryl sulfate.

The problem underlying the present invention is to provide an orally administered pharmaceutical solid form which releases an LTB₄ antagonist, in particular of formula I fast and completely and thus leads to better bioavailability of this active substance. A further object of the present invention is to prepare a formulation which is characterised by ease of handling during the preparation process and thus can be produced industrially in a reproducible manner while maintaining a constant high quality.

### SHORT DESCRIPTION OF THE INVENTION

Surprisingly it has been found that a solid pharmaceutical form comprising an LTB₄ antagonist, which is embedded in a polymer matrix (solid dispersion) obtainable by extrusion and shaping of a melt comprising a mixture of
(a) said LTB₄ antagonist;
(b) one or more fusible, pharmacologically acceptable polymer binders; and
(c) optionally one or more pharmaceutical auxiliaries, shows enhanced bioavailability.

Accordingly the invention relates to a a solid pharmaceutical form comprising an LTB₄ antagonist, which is embedded in a polymer matrix (solid dispersion) obtainable by extrusion and shaping of a melt comprising a mixture of said LTB₄ antagonist; one or more fusible, pharmacologically acceptable polymer binders; and optionally one or more pharmaceutical auxiliaries.

Another aspect of the invention is the use of such a solid pharmaceutical form for preparing a pharmaceutical composition for the treatment or prevention of diseases in which LTB₄ antagonists can be used therapeutically or preventively.

### SHORT DESCRIPTION OF THE DRAWINGS

FIG. 1 shows the blood plasma concentrations of an LTB₄ antagonist administered in the solid pharmaceutical form according to the present invention in comparison to the tablets disclosed by WO 03/007922.

### DETAILED DESCRIPTION OF THE INVENTION

Preferably the LTB₄ antagonists exhibit a benzamidino group of formula A, 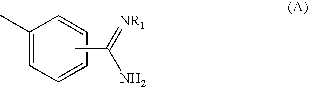
wherein R₁ represents a hydrogen atom or a group which is cleaved off under physiological conditions, particularly preferred are the compounds of formula I: 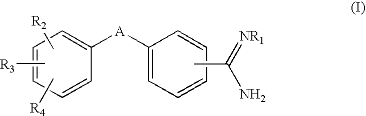
wherein
A denotes a group of formula
   —O—CₘH₂ₘ—O—(PHE)ₙ— (II)
   wherein
   m is an integer from 2 to 6, preferably 2 to 5,
   n is 0 or 1,
   PHE denotes a 1,4-phenylene group optionally substituted by one or two C₁-C₆ alkyl groups, preferably a 1,4-phenylene group substituted by a C₂-C₄ alkyl group in the ortho position linked to the oxygen; or
A denotes a group of formula 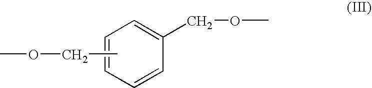
preferably of formula 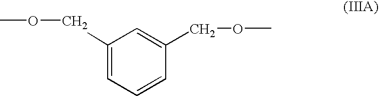
R₁ denotes H, OH, CN, COR₁₀, or CHO, preferably H or COOR₁₀;
R₂ denotes H, Br, Cl, F, CF₃, CHF₂, OH, HSO₃—O, C₁-C₆-alkyl, C₁-C₆-alkoxy, C₅-C₇-cycloalkyl, CONR₈R₉, aryl, O-aryl, CH₂-aryl, CR₅R₆-aryl, or C(CH₃)₂—R₇, preferably OH, HSO₃—O, CONR₈R₉ or CR₅R₆-aryl,
R₃ denotes H, C₁-C₆-alkyl, C₁-C₆-alkoxy, OH, Cl or F, preferably H or C₁-C₃-alkoxy,
R₄ denotes H or C₁-C₆-alkyl, preferably H;
R₅ denotes C₁-C₄-alkyl, CF₃, CH₂OH, COOH or COO(C₁-C₄-alkyl), preferably C₁-C₄-alkyl, particularly methyl;
R₆ denotes H, C₁-C₄-alkyl or CF₃, preferably C₁-C₄-alkyl, particularly methyl;
R₇ denotes CH₂OH, COOH, COO(C₁-C₄-alkyl), CONR₈R₉ or CH₂NR₈R₉;
R₈ denotes H, C₁-C₆-alkyl, phenyl, phenyl-(C₁-C₆-alkyl), COR₁₀, COOR₁₀, CHO, CONH₂, CONHR₁₀, SO₂—(C₁-C₆-alkyl), SO₂-phenyl, while the phenyl group may be mono- or disubstituted by Cl, F, CF₃, C₁-C₄-alkyl, OH and/or C₁-C₄-alkoxy, and preferably denotes C₁-C₄-alkyl, particularly isopropyl;
R₉ denotes H or C₁-C₆-alkyl, preferably H or C₁-C₄-alkyl, particularly isopropyl; or R₈ and R₉ taken together represent a C₄-C₆-alkylene group;
R₁₀ denotes C₁-C₆-alkyl, C₅-C₇-cycloalkyl, aryl, heteroaryl, aralkyl or heteroaryl-(C₁-C₆-alkyl), preferably C₁-C₄-alkyl,
   while the aryl groups mentioned in groups R₂ and R₁₀ denote phenyl or naphthyl, the heteroaryl groups denote pyrrole, pyrazole, imidazole, furanyl, thienyl, pyridine or pyrimidine and may each be mono- or polysubstituted by Cl, F, CF₃, C₁-C₄-alkyl, OH, HSO₃—O or C₁-C₄-alkoxy, preferably by OH or HSO₃—O—.

The active substance of formula I may be present in the formulation according to the invention in the form of a physiologically acceptable acid addition salt. By physiologically acceptable acid addition salts are meant, according to the invention, pharmaceutically acceptable salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. Mixtures of the above acids may also be used to prepare the salts. According to the invention, the preferred salts of formula I are selected from among the hydrochloride, hydrobromide, sulphate, phosphate, fumarate and methanesulphonate. The salts selected from among the hydrochloride, hydrobromide and fumarate are particularly preferred. The active substance may optionally be in the form of a hydrate. Preferably, according to the invention, the compound of formula I is added to the tablet in the form of the free base and in the anhydrous form.

Most preferred are the compounds of formulae IA, IB and IC, particularly IA:

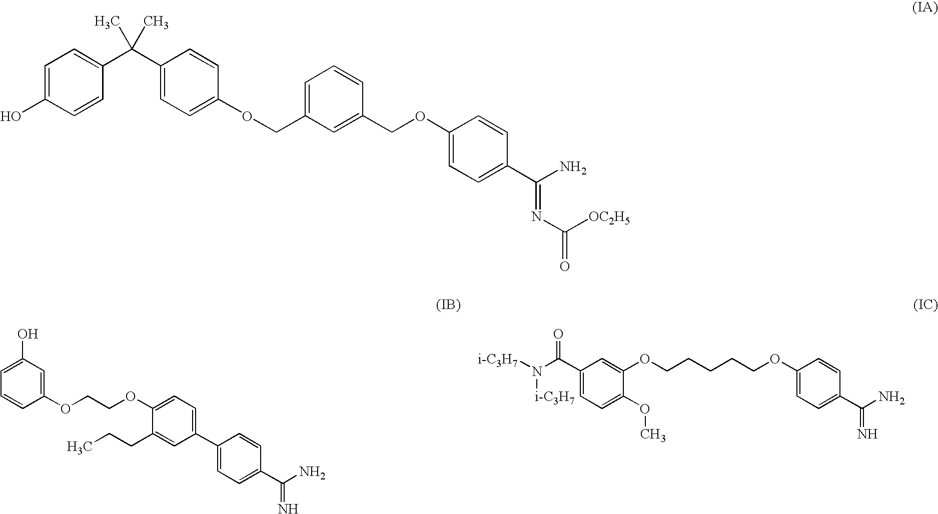

The compounds of formula I wherein R1 is different from hydrogen are generally prodrugs which are converted in vivo into the corresponding compounds of formula I wherein R1 is hydrogen.

For example, from the compound IA is formed in vivo the compound of formula IA1: 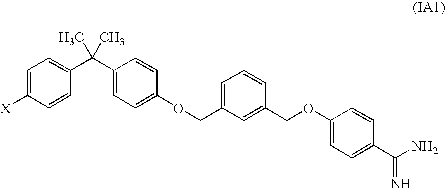
wherein X denotes OH, HSO₃—O or a carbohydrate group of formula C₆H₁₁O₅—O—.

Preferably, the active substance is used in crystalline, unground form or in ground form, particularly in jet-ground form, wherein the particle size distribution is within the following limits: D10≦3 μm, D50 3 to 8 μm, D90≦8 to 30 μm. The abovementioned numerical data for D10, D50 and D90 in μm (microns) are the particle size ranges within which a throughput total of 10 vol. %, 50 vol. % or 90 vol. % of the particles measured (cumulative volume distribution) is achieved. These values were determined by the laser diffractometry method, specifically, in the present instance, using a so-called dry dispersion under a dispersion pressure of 2 bar and with a focal length f=500 mm, e.g. using a Sympatec/RODOS apparatus. This methodology is known in the prior art.

Where reference is made to salts of the compounds of formula I within the scope of the present invention, this is indicated by the symbol I′. Explicit references to the free base of formula I, on the other hand, are indicated by the use of the symbol I.

In relation to the total mass of the solid form according to the invention the compound of formula I, particularly IA is present in an amount of up to 0.2 to 80 wt. %, preferably 0.7 to 40 wt. %, more preferably about 5 to 35 wt. %. Particularly preferred is a content of the free base of I between 6 and 30 wt. %, most preferred about 14.4 wt. % based on the total mass of the solid form.

The fusible, pharmacologically acceptable binder (b) is preferably selected from the group consisting of homopolymers of N-vinylpyrrolidone and water-soluble copolymers of N-vinylpyrrolidone. Preferably such polymers are essentially free of solvents.

The N-vinylpyrrolidinone (NVP) polymers should contain not less than 20, preferably not less than 60% by weight of NVP as copolymerized units and have a Fikentscher K value (Cellulose-Chemie 13 (1932), 58-64 and 71-74) of from 10 to 70, preferably from 10 to 50, particularly preferably from 12 to 40, in particular from 12 to 35 and, in the case of NVP homopolymers, preferably from 12 to 35, in particular from 12 to 17.

The polymeric binder must soften or melt in the total mixture of all components at from 50 to 180° C., preferably from 60° to 130° C., so that the melt can be extruded. The glass transition temperature of the mixture is preferably less than 180° C., in particular less than 130° C. If necessary, it is reduced by conventional pharmacologically acceptable plasticizers, such as long-chain alcohols, ethylene glycol, propylene glycol, triethylene gylcol, polyethylene glycols, aliphatic dicarboxylates (eg. dialkyl adipates, sebacates, citrates or tartrates) or fatty acid esters. The plasticizer preferably accounts for no more than 20% by weight, based on the polymer. Particularly preferred NVP polymers are those which do not require additives of this type, i.e. those which, as a mixture with the LTB₄ antagonist and, if required, conventional pharmaceutical auxiliaries, melt or soften in the desired temperature range even without additives having a specific plasticizing effect. Melting or softening below a certain temperature may be necessary because of possible thermal and/or oxidative damage not only to the active ingredient but also to the NVP polymer.

If the K value is greater than 17, in particular greater than 30 or even 40 (up to a maximum of 70), and no highly plasticizing component is present, the only suitable copolymers are those having a glass transition temperature T_{g} of less than 120° C., preferably less than 100° C.

Suitable comonomers are unsaturated carboxylic acids, e.g. methacrylic acid, crotonic acid, maleic acid and itaconic acid, and their esters with alcohols of 1 to 12, preferably 1 to 8, carbon atoms, as well as hydroxyethyl or hydroxypropyl acrylate and methacrylate, (meth)acrylamide, the anhydrides and half esters of maleic acid and itaconic acid (the half esters preferably not being formed until after the polymerization), N-vinylcaprolactam and vinyl propionate.

Preferred comonomers are acrylic acid and in particular vinyl acetate. Preferred NVP polymers are therefore those which either contain only NVP or vinyl acetate as the only comonomer or contain not less than 10, preferably not less than 30% by weight thereof as copolymerized units. Some or all of the vinyl acetate and vinyl propionate may be hydrolysed after the polymerization.

Preferably the pharmaceutical auxiliary (c) is selected from the group consisting of carriers, non-ionic emulsifiers and plasticizers, in particular from the group consisting of silicates, silica, stearic acid or salts thereof, methylcellulose, talc, sucrose, lactose, starch, polyethylene glycol esters of fatty acids, polysorbates, ethoxylated polysorbates, polyalkoxy alkoholates, alkylesters organic acids, in particular trialkyl citrates.

In a particularly preferred embodiment the pharmaceutical auxiliary (c) essentially consists of talc, glycerol-polyethylene glycol oxystearate and triethyl citrate.

Most preferred is a solid pharmaceutical form consisting essentially of
(a) an LTB₄ antagonist of formula (I), in particular formula (IA);
(b) a copolymer of N-vinylpyrrolidone and vinyl acetate; and
(c) talc, glycerol-polyethylene glycol oxystearate and triethyl citrate.

The active compound or compounds can be mixed with the binders and, where relevant, other conventional pharmaceutical additives before or after melting of the polymeric binder, by a method conventionally used in industry. Mixing is preferably carried out in an extruder having a mixing zone, preferably a twin-screw extruder, or in the screw zone of an injection molding machine. The melts obtained are essentially solvent-free. This means that no water or organic solvents are added unless the active compound is presented as a hydrate and/or a solvate.

Shaping may be effected by injection molding or by extrusion followed by shaping of the plastic extrudate, for example by hotface cutting to give granules or molding to give tablets, for example by passing the extrudate between two rollers which are driven in opposite directions and have depressions opposite one another in the roller shell, the form of these depressions determining the tablet shape. Cold-face cutting is also suitable and may be followed by pressing of the granules to give tablets. For the purpose of the present invention, the term extrusion includes injection molding. The shaped extrudates have a content of residual organic solvent of less than 0.1% by weight. Solvates of the active compound are not addressed with this statement.

In the pharmaceutical composition according to the invention the active ingredient is present as a solid dispersion.

The term “solid dispersion” as used hereinbefore or hereinbelow is understood to mean a finely dispersed distribution of one or more solids in an inert solid or semi-solid carrier. The active ingredient may be present in molecular dispersed form, i.e. as a solid solution, in fine crystalline dispersed form, in a glassy amorphous phase or dispersed as a fine amorphous powder. Eutectic mixtures, i.e. crystalline structures of actives substances and carriers are also encompassed in this definition.

By varying the type and amount of comonomer, the NVP polymer can, depending on the intended use, be made sufficiently strongly or weakly hydrophilic for the tablets prepared from it to dissolve (rapidly or with a delay) in the mouth (buccal tablets) or in the stomach or not until they reach the intestine, or to swell so that they release the active compound. They are sufficiently swellable when they absorb more than 10% by weight of water on storage at 90% relative humidity. If it is desirable for carboxyl-containing binders to release the active compound only when they reach the alkaline medium of the intestine, the above water absorption applies only to the neutralized form (salt form) of the polymer (in which some or all of the protons of the carboxyl groups have been replaced by ammonium, sodium or potassium ions).

If desired, the solid pharmaceutical form may also be provided with a conventional coating to improve the appearance and/or the flavor (coated tablets) or additionally to delay the release of active compound. For oral tablets with sustained release of active compound, it may be advantageous to prepare the tablet by one of the known techniques in a closed-cell porous form so that it floats in the stomach and consequently remains there longer.

In the case of solid pharmaceutical forms with rapid release of active compound, the novel process permits substantially freer design of the pharmaceutical form than does the conventional tablet pressing technique. For example, the tablets can be engraved for designation, or virtually any shapes, which are clearly identifiable even by those with impaired vision, may be produced. Certain shapes, for example hemispheres may also be suitable for achieving certain characteristics of active compound release. By extrusion or hot or cold face cutting of the extrudate, it is possible to produce very small-particled and uniformly shaped granules in a simple manner, for example for multiple-unit forms.

In the Examples which follow, parts and percentages are by weight. The active compound release time was determined by the half-change test method.

### EXAMPLE 1

66.7 parts per weight of a copolymer of 60% by weight of N-vinylpyrrolidone and 40% by weight of vinyl acetate, having a K value of 30, 1.5 parts per weight of triethyl citrate, 12 parts of Cremophor® RH40 (glycerol-polyethylene glycol oxystearate commercially available from BASF AG, Germany), 5.8 parts per weight of talc and 14.4 parts of compound of formula (IA) were processed to tablet cores in a twin-screw extruder at 100° C. Immediately after leaving the extruder, the hot melt was shaped into oblong tablets by calendering. The tablet cores obtained were stable to mechanical effects and did not show any abrasion during transportation and packaging. In the half-change test (cf. for example R. Voigt, Lehrbuch der pharmazeut. Technologie, 5th Edition, Verl. Chemie. Weinheim; Deerfield Beach, Fla.; Basel, 1984, page 627) in conjunction with the paddle method according to USP 21, the active compound was completely released in the course of from 6 to 8 hours.

The conventional compressed tablet described in WO 03/007922 consists of crystalline compound of formula (IA), Avicel-PH101, lactose-H₂O, sodium lauryl sulfate, Kollidon-CL and magnesium stearate was compared with the dosage form of example 1. Both tablets were tested in a four way cross over, randomised study with 16 healthy, male volunteers. Single doses of 75 mg were administered under fed and fasted conditions (wash out phase: at least 6 days). The glucoronidised metabolite of formula (IA) was used as analyte to monitor plasma concentrations.

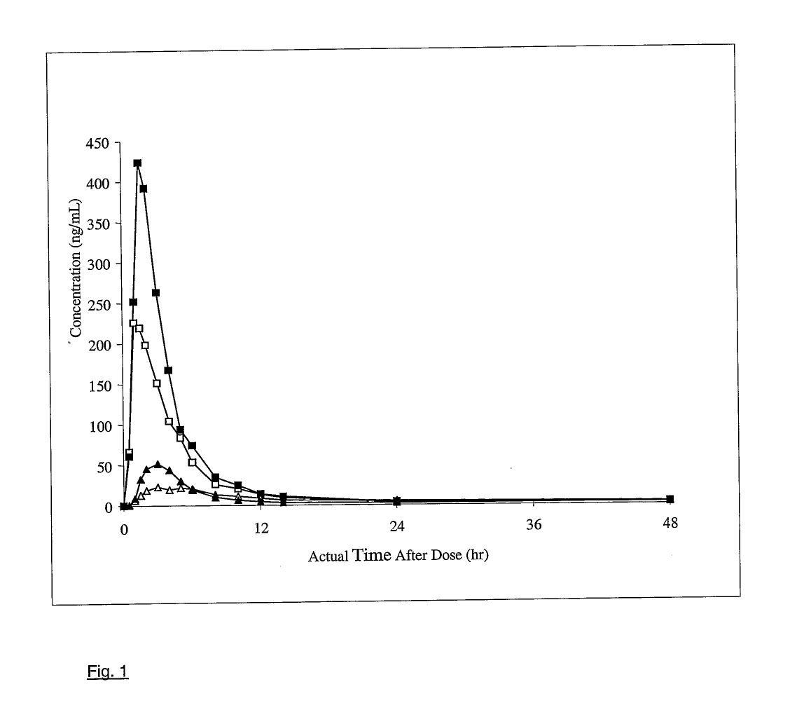

The blood plasma concentration obtained with these tablets are shown in FIG. 1, in which the graphs have the following meanings:
-□-composition of Example 1, fasted conditions
-▪-composition of Example 1, fed conditions
-Δ-composition of WO 03/007922, fasted conditions
-▴-composition of WO 03/007922, fed conditions

The high surface area provided by the solid dispersion formulation of example 1 facilitated/supported drug absorption and in consequence enhanced oral bioavailability. Additionally, the observed food effect was lower for the tablet of the invention (factor 1.6) compared to the compressed tablet of WO 03/007922 (factor 2.0) and variability was reduced significantly under fed conditions for the inventive tablet.

Conclusions. The formulation of formula (IA) as a stable solid dispersion by melt extrusion technology led to increased oral bioavailability and thus improved in vivo performance. X-ray diffraction of the formulation showed that formula IA existed as a molecular dispersion in the matrix polymer.

1. A solid pharmaceutical form comprising an LTB₄ antagonist, which is embedded in a polymer matrix (solid dispersion) obtainable by extrusion and shaping of a melt comprising a mixture of
(a) an LTB₄ antagonist;
(b) one or more fusible, pharmacologically acceptable polymer binders; and
(c) optionally one or more pharmaceutical auxiliaries.

2. A solid pharmaceutical form according to claim 1, wherein the LTB₄ antagonist is a compound of formula I, 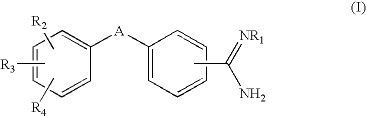
wherein
A denotes a group of formula
—O—CₘH₂ₘ—O—(PHE)ₙ— (II)
wherein
m is an integer from 2 to 6,
n is 0or 1,
PHE denotes a 1,4-phenylene group optionally substituted by one or two C₁-C₆ alkyl groups; or
A denotes a group of formula 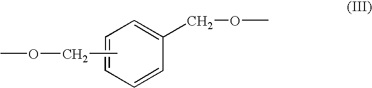
R₁ denotes H, OH, CN, COR₁₀, or CHO;
R₂ denotes H, Br, Cl , F, CF₃, CHF₂, OH, HSO₃—O, C₁-C₆-alkyl, C₁-C₆-alkoxy, C₅-C₇-cycloalkyl, CONR₈R₉, aryl, O-aryl, CH₂-aryl, CR₅R₆-aryl, or C(CH₃)₂—R₇,
R₃ denotes H, C₁-C₆-alkyl, C₁-C₆-alkoxy, OH, Cl or F,
R₄ denotes H or C₁-C₆-alkyl;
R₅ denotes C₁-C₄-alkyl, CF₃, CH₂OH, COOH or COO(C₁-C₄-alkyl);
R₆ denotes H, C₁-C₄-alkyl or CF₃;
R₇ denotes CH₂OH, COOH, COO(C₁-C₄-alkyl), CONR₈R₉ or CH₂NR₈R₉;
R₈ denotes H, C₁-C₆-alkyl, phenyl, phenyl-(C₁-C₆-alkyl), COR₁₀, COOR₁₀, CHO, CONH₂, CONHR₁₀, SO₂—(C₁-C₆-alkyl), SO₂-phenyl, while the phenyl group may be mono- or disubstituted by Cl, F, CF₃, C₁-C₄-alkyl, OH and/or C₁-C₄-alkoxy;
R₉ denotes H or C₁-C₆-alkyl; or
R₈ and R₉ taken together represent a C₄-C₆-alkylene group;
R₁₀ denotes C₁-C₆-alkyl, C₅-C₇-cycloalkyl, aryl, heteroaryl, aralkyl or heteroaryl-(C₁-C₆-alkyl),
while the aryl groups mentioned in groups R₂ and R₁₀ denote phenyl or naphthyl, the heteroaryl groups denote pyrrole, pyrazole, imidazole, furanyl, thienyl, pyridine or pyrimidine and may each be mono- or polysubstituted by Cl, F, CF₃, C₁-C₄-alkyl, OH, HSO₃—O or C₁-C₄-alkoxy, as well as the pharmacologically acceptable acid addition salts and glycosides and O-sulphates thereof.

3. A solid pharmaceutical form according to claim 2, wherein the LTB₄ antagonist is selected from among formulae IA, IB and IC:

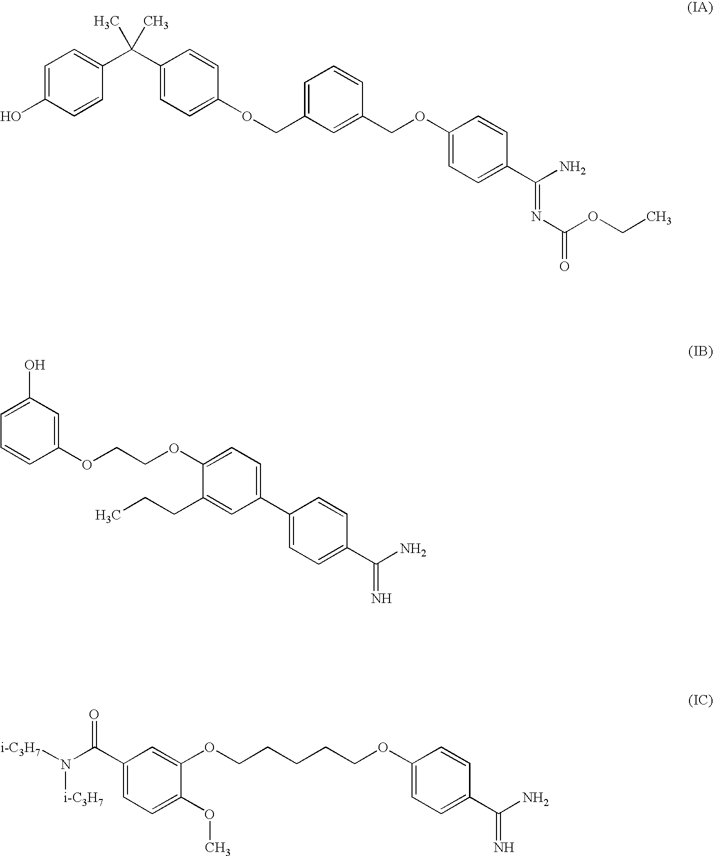

4. A solid pharmaceutical form according to claim 3, wherein said fusible, pharmacologically acceptable binder (b) is selected from the group consisting of homopolymers of N-vinylpyrrolidone and water-soluble copolymers of N-vinylpyrrolidone.

5. A solid pharmaceutical form according to claim 4, wherein said fusible, pharmacologically acceptable binder (b) is a copolymer of N-vinylpyrrolidone and vinyl acetate.

6. A solid pharmaceutical form according to claim 5, wherein said pharmaceutical auxiliary (c) is selected from the group consisting of carriers, non-ionic emulsifiers and plastisizers.

7. A solid pharmaceutical form according to claim 6, wherein said pharmaceutical auxiliary (c) is selected from the group consisting of silicates, silica, stearic acid or salts thereof, methylcellulose, talc, sucrose, lactose, starch, polyethylene glycol esters of fatty acids, polysorbates, ethoxylated polysorbates, polyalkoxy alkoholates, and alkyl esters of organic acids.

8. A solid pharmaceutical form according to claim 7, wherein said pharmaceutical auxiliary (c) essentially consists of talc, glycerol-polyethylene glycol oxystearate and triethyl citrate.

9. A solid pharmaceutical form according to claim 8 consisting essentially of
(a) an LTB₄ antagonist of formula (I);
(b) a copolymer of N-vinylpyrrolidone and vinyl acetate; and
(c) talc, glycerol-polyethylene glycol oxystearate and triethyl citrate.

10. A solid pharmaceutical form according to claim 9, which is obtainable by melt extrusion on a 18 mm twin-screw extruder.

11. A method of treating a disease in which LTB₄ is involved comprising administering to a patient a therapeutically effective amount of a solid pharmaceutical form according to claim 1.

12. A method of treating a disease chose from arthritis, asthma, chronic obstructive pulmonary diseases, psoriasis, ulcerative colitis, Alzheimer's disease, shock, reperfusion damage/ischaemia, cystic fibrosis, atherosclerosis and multiple sclerosis comprising administering to a patient a therapeutically effective amount of a solid pharmaceutical form according to claim 1.
